(19) United States
(12) Patent Application Publication
(10) Pub. No.: US 2006/0193902 A1
(43) Pub. Date: Aug. 31, 2006

(54) Pharmaceutical compositions containing active agents having a lactone group and transition metal ions

(75) Inventors: Paul Tardi, Surrey (CA); Sharon Johnstone, Vancouver, CA (US)

Correspondence Address: MORRISON & FOERSTER LLP, 12531 HIGH BLUFF DRIVE, SUITE 100, SAN DIEGO, CA, 92130-2040, US

(73) Assignee: Celator Pharmaceuticals, Inc., Princeton, NJ (US)

(21) Appl. No.: 10/551,572

(22) PCT Filed: Apr. 2, 2004
(86) PCT No.: PCT/CA04/00505

§ 371 Date: Mar. 31, 2006

Related U.S. Application Data
(60) Provisional application No. 60/460,171, filed on Apr. 2, 2003.

(51) Int. Cl. A61K 31/4745; A61K 9/127

(52) U.S. Cl. 424/450; 514/283; 977/907

(57) ABSTRACT

Compositions and methods for stabilizing an active agent containing one or more actone rings. The compositions, including pharmaceutical compositions, ensure that the lactone ring of the active agent is stabilized in the active, ring-closed form due to the inclusion of a transition metal ion.

### CROSS-REFERENCE TO RELATED APPLICATIONS

This application claims benefit under 35 U.S.C. § 119(e) of U.S. Ser. No. 60/460,171 filed 2 Apr. 2003, which is hereby incorporated by reference in its entirety.

### TECHNICAL FIELD

This invention relates to compositions and methods for stabilizing an active agent containing one or more lactone rings. More particularly, the invention concerns a pharmaceutical preparation that ensures that the lactone ring of the active agent is stabilized in the active, ring-closed form due to the inclusion of a transition metal ion. Optionally, the active agent-metal complex is stably associated with a delivery vehicle to allow for enhanced delivery of the active agent to a target site.

### BACKGROUND OF THE INVENTION

Camptothecin is a plant-derived alkaloid that is effective in cancer chemotherapy by interfering with the breakage/reunion actions of nuclear topoisomerase I. This inhibitory effect is believed to result from the binding of the drug to topoisomerase I-DNA adducts. The inhibition of this enzyme induces single-strand nicks in DNA, which causes arrest in the G2 phase of the cell cycle. It has been shown that camptothecin exhibits cytotoxicity in human malignant tumors xenografted in immunodeficient nude mice (Giovanella, et al., Cancer Res (1991) 51:3052-5, Giovanella, et al., Science (1989) 246:1046-1048, Pantazis, et al., Cancer Res (1993) 53:1577-82, Pantazis, et al., Cancer Res 52:3980-7, Pantazis, et al., Int J Cancer (1993) 53:863-71).

The development of camptothecin as a pharmaceutical agent has been limited due to its water insolubility, thus making it difficult to formulate the drug as well as deliver it to target cells. In recent years, various water-soluble derivatives of camptothecin have been synthesized including irinotecan (camptothecin-11), topotecan and lurtotecan with the goal of increasing the formulation of the drugs. Irinotecan has been approved as a treatment for metastatic cancer of the colon or rectum and is commonly prescribed in colorectal cancer cases that have not responded to standard treatment with fluorouracil. However, despite promising results in the clinic, irinotecan has exhibited lethal side effects such as myelosuppression and gastrointestinal disorders (Nishimura, et al., Jpn. J. Cancer Chemother (1995) 22:93-97, Ohe, et al., J. Natl. Cancer Inst. (1992) 84:972-974 and Takasago, et al., Folia Pharmacol. Jpn (1995) 105:447-460). The FDA has approved topotecan as a treatment for advanced ovarian cancers that have resisted other chemotherapy drugs. However, topotecan has also been shown to induce myelosuppression, which is characterized by brief and noncumulative neutropenia (Slichenmyer, et al., Journal of the National Cancer Institute (1993) 85:271-290). Researchers have turned to formulation of these drugs into liposomes with the goal of decreasing their toxicity while at the same time maintaining anti-tumour activity.

An additional drawback to the use of camptothecin and related analogs is the tendency of the drugs to undergo rapid hydrolysis in the blood shortly after administration. This is due to the presence of an α-hydroxyl lactone ring which equilibrates between a ring-open carboxylate form and a closed lactone form. The carboxylate form of the drug is known to be poorly accumulated in cancer cells, possibly due to the inability of this form of the drug to cross cell membranes; therefore, the closed lactone ring is important for maintaining cytotoxic activity. As well, studies with camptothecin have shown that the open-ring form is a less potent inhibitor of topoisomerase I than the non-hydrolyzed form (Hertzberg, et al., J. Med Chem (1989) 32:715-720 and Jaxel, et al., Cancer Res. (1989) 49:1465-1469). The activity of camptothecin can be abolished by substitution of the oxygen of the lactone ring with sulfur or nitrogen, thus further supporting the notion that it is essential to the activity of the drug (Hertzberg, et al., (supra) and Jaxel, et al., (supra)). The equilibrium constant for this reaction is pH-dependent with 90% of the compound being present in the lactone form at pH 4.5 and 10% being present in the lactone form at pH 7.4 (Slichenmyer, et al., Journal of the National Cancer Institute (1993) 85:271-290). Thus the utility of camptothecins and related analogs has been limited as, at physiological pH, the drug equates toward the inactive carboxylate form. It has been reported that this conversion occurs rapidly in the blood with only 5% of the lactone ring of camptothecin being present following 3 hours of incubation in human blood (Bom et al., Proceedings of the 1999 AACR, NCI, EORTC International Conference).

In addition to camptothecins, other anti-cancer drugs such as bryostatin and rhizoxin contain a lactone ring. Bryostatin, a cytotoxic agent derived from a single-cell sea organism, has both cytotoxic and immunomodulatory properties in in-vivo and in-vitro models. Recently, it has been suggested that bryostatin has a role in the treatment of renal cell carcinoma in Phase II clinical trials (Haas, et al., Clin Cancer Res (2003) 9(1):109-14). Another macrocyclic lactone, rhizoxin, has been isolated from the plant pathogenic fungus Rhizopus chinesis and has been shown to inhibit angiogenesis. It has been suggested that rhizoxin exerts antiangiogenic effects by inhibiting functions of endothelial cells responsible for induction of in vivo angiogenesis (Aoki, et al., Eur J Pharmacol (2003) 459(2-3):131-8). Furthermore, various antibiotics are known to contain a lactone ring.

In an attempt to stabilize camptothecins in the active state after intravenous administration, liposomal encapsulation using pH gradients has been employed (see Slater, et al., U.S. Pat. No. 6,355,268). This technique involves preparing pre-formed liposomes such that the internal aqueous solution is at a reduced pH (generally around 4.0) with respect to the external solution. After establishment of a transmembrane pH gradient, drug is added to the extraliposomal solution and uptake occurs due to conversion of the drug from its neutral form at neutral pH to its charged form at reduced pH (see Mayer, et al., U.S. Pat. Nos. 6,083,530, 5,795,589, 5,616,341 and 5,744,158). Formulation of the lactone drug at a reduced pH allows for the stabilization of the drug in the ring-closed form. A disadvantage of this approach is that low intraliposomal pH conditions may not be suitable for long-term storage due to degradation of lipids in a low pH environment. A further limitation inherent to this approach is that conventional techniques utilized to load ionizable agents in response to a pH gradient are not suitable for loading camptothecin drugs such as irinotecan. As well, since the transmembrane pH gradient can only be maintained for short periods of time, clinical formulation of drugs into liposomes requires the generation of a proton gradient just prior to drug loading.

Another approach to preserve the activity of camptothecin drugs involves stabilizing the lactone ring by intercalation into liposomal membranes (see Burke, et al., U.S. Pat. No. 5,552,156). As disclosed by Burke, et al., (supra) this technique involves preparing liposomes in neutral pH followed by incubation with drug. Insertion of the lactone ring into lipid bilayers was reported to protect the drug from ring hydrolysis as measured by HPLC. These results were supplemented with drug permeation studies, which indicated that increases in steady-state fluorescent anisotropy were observed due to relocation of camptothecin from an aqueous environment to the lipid membrane. A drawback of the techniques described in this patent is the requirement to employ low drug-to-lipid ratios in order to achieve high encapsulation efficiency. Such low drug-to-lipid ratios make it difficult to achieve a sufficient drug load in the liposomes for clinical efficacy.

The present invention is based on the finding that active agents requiring an intact lactone ring for activity can be stabilized against hydrolysis by the presence of a transition metal ion. An advantage of this method of stabilization is that it may be performed at physiologically-relevant pH ranges that normally result in conversion to the biologically less active carboxylate form of the lactone ring. This alleviates the need to employ low pH values commonly utilized to actively load drugs into liposomes. By maintaining the ring-closed form of the drug, the activity of the lactone-containing active agent can be stably delivered to a target site. Optionally, metal/active agent complexes of the present invention may be incorporated into delivery vehicles to further enhance stable delivery of the lactone drug to a target site and to reduce toxicity of the lactone-containing agent to non-target cells.

Kuwahara, et al., in Biochemistry (1986) 25:1216-1221 and Nucleic Acids Symp Ser. (1985) 16:201-204 have examined the ability of camptothecin-copper complexes to produce single- and double-strand breaks of DNA upon irradiation with 365 nm light. The investigators suggest that Cu(II) ion may act as a cofactor in the antitumour action of camptothecin when combined with photochemotherapy due to the ability of the drug-metal complex to generate free radicals that lead to DNA damage. Although the results presented in these papers demonstrate that copper specific promotion of DNA cleavage occurred, the ability of the metal ion to stabilize the lactone form of the drug was not suggested. Additionally, the investigation was limited only to camptothecin, and water-soluble analogs were not evaluated for their ability to cleave DNA. Furthermore, although concentrations of the metal-drug complex employed were suitable for DNA cleavage studies, these concentrations would not be suitable for pharmaceutical preparations.

Hertzberg, et al., Biochemistry (1989) 28:4629-4638 also examined U-v-light-induced cleavage of Cu(II)-camptothecin as well as copper(II) complexes of the 20-deoxy and 10-hydroxy camptothecin derivatives. In the presence of long-wave UV light, camptothecin and 20-deoxycamptothecin complexes exhibited 51% and 28% DNA cleavage respectively, while the 10-hydroxycamptothecin derivative complex only cleaved 1.7% of the DNA. Concentrations of CuSO₄ and camptothecin used in these studies were only 10 μM for both the drug and the metal. As with the Kuwahara et al. 1985 and 1986 (supra) studies, the ability of the metal ion to stabilize the lactone form of the drugs was not suggested.

### DISCLOSURE OF THE INVENTION

The pharmaceutical preparation described herein provide for the enhanced stability of an active agent containing a lactone ring. The pharmaceutical preparations contain one or more transition metal ions that ensure that the activity of active agent is maintained under conditions in which the lactone ring is normally unstable due to high levels of hydrolysis. Hydrolysis of the lactone ring has been correlated with inactivity and thus it is desirable to ensure that the drug equates to the ring-closed, non-hydrolyzed form. This invention overcomes difficulties previously encountered in the art to stabilize the lactone form of an agent, such as the requirement for a low pH environment or the incorporation of the lactone moiety into a lipid bilayer. Although many active agents containing lactone rings are anti-cancer agents, the use of these agents is not limited to cancer treatment as many other active agents, such as antibiotics, contain a lactone moiety. In order to further enhance stable delivery to a target site, the metal-active agent preparation may optionally be stably associated with a delivery vehicle. This allows for the stable delivery of the complex by altering the pharmacokinetics of the preparation after administration to a subject.

Thus, in one aspect, the invention is directed to a pharmaceutical preparation comprising an active agent having a lactone ring and a transition metal ion in sufficient concentration to decrease the percentage of the lactone ring in the ring-open form relative to preparations of the agent lacking a metal ion. Preferably, the active agent is a water-soluble camptothecin analog such as topotecan, lurtotecan or irinotecan.

In a preferred embodiment, the pharmaceutical preparation is stably associated with a delivery vehicle. Any suitable delivery vehicle may be utilized, including lipid carriers, liposomes, lipid micelles, lipoprotein micelles, polymer nanoparticles, polymer-lipid hybrid systems and the like. Preferred delivery vehicles are nanoparticles and liposomes.

This invention further provides methods of administering the pharmaceutical preparation to a mammal, and methods of treating a mammal affected by or susceptible to or suspected of being affected by a disorder (e.g., cancer).

### BRIEF DESCRIPTION OF THE DRAWINGS

FIG. 1: A graph showing loading of irinotecan into DSPC/DSPG/Chol (7:2:1 mole ratio) liposomes as a function of time using copper, zinc or manganese gluconate buffered with triethanoloamine (TEA) as the internal medium. Loading was carried out at 50° C. at a drug-to-lipid mole ratio of 0.1:1.

FIG. 2: A graph showing loading of irinotecan into DSPC/DSPG/Chol (70:20:10 mole ratio) liposomes as a function of time using 100 mM Cu(II)gluconate buffered to pH 7.4 with 220 mM triethanolamine (TEA) as the internal medium and 20 mM HEPES, 150 mM NaCl, pH 7.45 (HBS), pH 7.4 as the external medium. Loading was carried out at 50° C. at a drug-to-lipid mole ratio of 0.1:1.

FIG. 3: A graph showing loading of irinotecan into DPPC/Chol (55:45 mole ratio) liposomes as a function of time using 100 mM Cu(II)gluconate adjusted to pH 7.4 with TEA as the internal medium and SHE, pH 7.4 as the external medium. Loading was carried out at 50° C. at a drug-to-lipid weight ratio of 0.1:1.

FIG. 4A: A graph showing loading of irinotecan into Floxuridine (FUDR)-containing DSPC/DSPG/Chol (70:20:10 mole ratio) liposomes as a function of time using 100 mM Cu(II)gluconate, 220 mM TEA, pH 7.4 as the internal medium and 300 mM sucrose, 20 mM HEPES, pH 7.4 as the external buffer. FUDR was passively encapsulated and irinotecan loading was carried out at 50° C. at a drug-to-lipid mole of 0.09:1.

FIG. 4B: A graph showing loading of irinotecan into FUDR containing DSPC/DSPG liposomes at an 85:15 mole ratio as a function of time using 100 mM Cu(II)gluconate, 220 mM TEA, pH 7.4 as the internal medium and 300 mM sucrose, 20 mM HEPES, pH 7.4 as the external solution. FUDR was passively encapsulated and irinotecan loading was carried out at 50° C. at a drug-to-lipid mole ratio of 0.1:1.

FIG. 5A: Thin layer chromatography (TLC) of liposomal formulations of irinotecan and of aqueous irinotecan solutions that were incubated in buffers ranging between pH 2 and 9. The upper lactone and lower carboxylate band were visualized by UV light.

FIG. 5B: HPLC analysis of the carboxylate and lactone forms of liposomal irinotecan loaded with copper sulfate (Lane A of FIG. 5A).

### MODES FOR CARRYING OUT THE INVENTION^{I}

The invention provides pharmaceutical preparations that are useful in reducing the hydrolysis of an active agent containing a lactone ring by the inclusion of a transition metal ion in the preparation. The transition metal ion is preferably selected to form a coordination complex with the active agent to promote maintenance of the ring-closed form of the lactone ring. Formation of the complex may occur through the oxygen coordination sites on the lactone ring thereby preventing formation of the carboxylate form of the drug. Preferred metal ions for complexation include those of Zn, Cu or Co.
Abbreviations: EDTA: ethylenediaminetetraacetic acid; HEPES: N-[2-hydroxylethyl]-piperazine-N-[2-ethanesulfonic acid]; HBS: HEPES buffered saline (20 mM HEPES, 150 mM NaCl, pH 7.4); SHE: 300 mM sucrose, 20 mM HEPES, 30 mM EDTA; TEA: triethanolamine; HPLC: high performance liquid chromotography Chol: cholesterol; DSPC: distearoylphosphatidylcholine; DPPC: dipalmitoylphosphatidylcholine; DSPG: distearoylphosphatidylglycerol; MLV: multilamellar vesicle; LUV: large unilamellar vesicle.

Preferred active agents are camptothecins and related analogs, although non-camptothecin drugs containing a lactone moiety may also be employed, such as bryostatin and rhizoxin as well as antibiotics. Lactone-containing agents for use in this invention are those in which the ring-closed form of the lactone moiety is optimal for therapeutic activity. The active agent is may be for example, a water-soluble camptothecin analog such as topotecan, irinotecan or lurtotecan.

In preferred embodiments, the pH of the pharmaceutical preparation is around physiological pH. In the absence of a metal ion, 90% of the lactone ring is present in the carboxylate form at pH 7.4 (Slichenmyer, et al., J. Natl. Cancer Inst. (1993) 85:271-291). This invention allows for the stable preparation of the active agent at a pH in the range of physiological pH.

In a preferred embodiment, the lactone-containing active agent and the transition metal are stably associated with a delivery vehicle. Particularly suitable delivery vehicles include liposomes and polymer nanoparticles, although other carriers may be used as well. The metal may be complexed to a component of the delivery vehicle, such as a lipid head group containing a chelation group. Polymer-metal-drug complexes may be incorporated into the polymeric matrix of nanoparticles and microparticles by the use of polymers containing coordination sites as further described herein.

Lactone Containing Active Agents

Suitable lactone-containing agents for use in this invention are camptothecins and related analogs. Members of the camptothecin class of compounds have the same core ring structure as given below. The lactone ring is denoted by ring E and complexation with transition metals may occur through the oxygen coordination sites as denoted in the figure below:

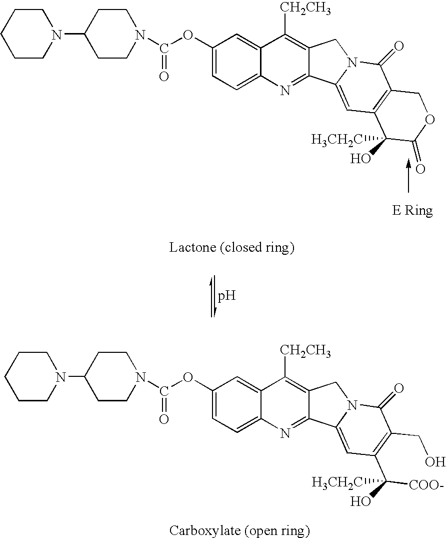

Camptothecin analogs that maintain anti-tumour activity are generally prepared by modifying ring A and B of the basic drug structure. For instance, the water-soluble camptothecin derivative, irinotecan, is characterized by a piperidino group attached to ring A. Camptothecin analogs created by addition of a hydrophilic hydroxyl or nitro group at the 9, 10 or 11 positions of ring A have been shown to exhibit enhanced solubility in aqueous solutions (Hsiang, et al., Cancer Res. (1989) 49:4385-4389, Jaxel, et al., (supra), Kingsbury, et al., J. Med. Chem. (1991) 34:98-107). Non-limiting examples of suitable camptothecin analogs that may be used in this invention include irinotecan, lurtotecan, topotecan, 9-aminocamptothecin, 9-nitrocamptothecin; 10-hydroxycamptothecin, 10,11-methylenedioxycamptothecin, 9-chloro-10,11-methylenedioxycamptothecin and 9-amino-10,11-methylene-dioxycamptothecin, 7-ethylcamptothecin and 20-deoxycamptothecin. In addition, various silicon derivatives of camptothecin have also been described in Bom et al. Journal of Controlled Release (2001) 74:325-333 and may be used in the practice of this invention.

Preferably, the camptothecin is a water-soluble analog. This may include an analog that is charged when in the physiological pH range. Examples of camptothecin analogs that are charged at physiological pH include topotecan, lurtotecan and irinotecan. This charge is due to the presence of groups on rings A and B in the structure above rather than to the carboxylate group of the lactone ring which is deprotonated in the ring-open form at physiological pH.

Transition Metals for Lactone Stabilization

Transition metal ions are those recognized in the art that occupy positions in the periodic chart between the alkaline earths and the column headed by B and Al—i.e. mainly atomic numbers 21-30, 39-48, and those in the same columns. Suitable ions include, for instance, those formed from Fe, Co, Ni, Cu, Zn, V, Ti, Cr, Rh, Ru, Mo, Mn and Pd. Preferably, the ion is Zn+2, Co+3, or Cu+2, most preferably Cu+2. Various salts of transition metals that are pharmaceutically acceptable and soluble in aqueous solvent may be utilized. Examples of suitable salts include chlorides, sulfates, tartrates, citrates, phosphates, nitrates, carbonates, acetates, glutamates, gluconates, glycinates, histidinates, lysinates and the like. An example of metal loading using a gluconate salt is provided in Example 1.

The concentration of the metal ion or lactone-agent in the preparation is preferably greater than 100 μM when administered in the free form. If the active agent or metal is administered at concentrations below 100 μM, the therapeutic effectiveness of the lactone drug may be too low to be of any utility. A preferred range is from 500 μM to 200 mM. The concentration of metal ion or lactone-containing agent when encapsulated in a delivery vehicle such as a liposome is preferably from 30 mM to about 500 mM and more preferably from about 50 to about 350 mM. The metal/agent complex may be suspended in a suitable buffer that is preferably within the physiological pH range.

Preferably, the metal ion/agent preparation is suspended in a metal compatible solution. A metal compatible solution is one that consists of a metal in solution that does not cause precipitation to occur for at least the time required to prepare and administer the pharmaceutical preparations. Preferably, the metal solution should be clear and soluble, free of aggregation, precipitation or flocculation for at least about 4 hours. By way of example, a 300 mM solution of MnSO₄ in pH 7.4 HEPES buffer is not a metal compatible solution as it produces an obvious brown precipitate of Mn(OH)₂ comprising approximately 6-7 mole % of the manganese added to the solution.

Measurement of the relative amounts of the lactone and hydroxy form of a lactone-containing agent may be determined using standard techniques known in the art. A particularly preferred technique is HPLC analysis and may be carried out as set forth in Example 5. Protection of the lactone ring from hydrolysis in accordance with this invention refers to the stabilization of the ring-closed form of a lactone-containing agent such that a higher level of the lactone form of the drug is present in the presence of a metal ion relative to the absence of the metal ion. The pH of the pharmaceutical preparation is preferably about 6.0 to about 8.0; most preferably, the pH is physiological pH (7.4). The percentage of the active agent present in the lactone form, within the physiological range, after addition of a transition metal is preferably greater than 20 mole %, most preferably greater than 40 mole % and even more preferably greater than 50 mole %. These measurements are preferably conducted at 37° C. at physiological pH and at 3 hours after incubation, most preferably at 24 hours after incubation. Suitable experimental conditions are set forth in Example 2.

Delivery Vehicles

Optionally, the metal/active agent preparation may be stably associated with one or more delivery vehicles. Delivery vehicles for use in this invention include lipid carriers, liposomes, lipid micelles, lipoprotein micelles, lipid-stabilized emulsions, cyclodextrins, polymer nanoparticles, polymer microparticles, block copolymer micelles, polymer-lipid hybrid systems, derivatized single chain polymers, and the like.

A particularly suitable delivery vehicle for use in this invention is a liposome. Liposomes can be prepared as described in Liposomes: Rational Design (A. S. Janoff ed., Marcel Dekker, Inc., N.Y.), or by additional techniques known to those knowledgeable in the art. Examples of liposomes for use in this invention include large unilamellar vesicles (LUVs), multilamellar vesicles (MLVs), small unilamellar vesicles (SUVs) and interdigitating fusion liposomes. Liposomes may comprise surface stabilizing hydrophilic polymer-lipid conjugates such as polyethylene glycol-DSPE, to enhance circulation longevity.

Negatively charged lipids such as phosphatidylglycerol (PG) and phosphatidylinositol (PI) can be included in liposomal formulations to increase the circulation longevity of the carrier as well. These lipids may be employed to replace hydrophilic polymer-lipid conjugates as surface stabilizing agents. Liposomes of the invention may also contain therapeutic lipids such as bioactive sphingolipids. Further examples include ether lipids, phosphatidic acid, phosphonates and phosphatidylserine.

Various methods may also be utilized to encapsulate active agents containing a lactone ring in liposomes. Examples of loading techniques include conventional passive and active entrapment methods. Passive methods of encapsulating active agents in liposomes involve encapsulating the agent during the preparation of the liposomes. This includes a passive entrapment method described by Bangham, et al., (J. Mol. Biol. (1965) 12:238). This technique results in the formation of multilamellar vesicles (MLVs) that can be converted to large unilamellar vesicles (LUVs) or small unilamellar vesicles (SUVs) upon extrusion. Additional methods of passive encapsulation include an ether injection technique described by Deamer and Bangham (Biochim. Biophys. Acta (1976) 443:629) and the Reverse Phase Evaporation technique as described by Szoka and Paphadjopoulos (P.N.A.S. (1978) 75:4194).

A technique employing encapsulated transition metals to drive the uptake of drugs into liposomes may be used in this invention. Drug entrapment according to this method relies on the formation of a drug-metal complex to drive uptake of a drug. The technique first involves preparing liposomes with an encapsulated transition metal by conventional passive loading techniques. A preferred passive loading technique involves first combining lipids in an organic solvent such as chloroform to give a desired mole ratio. The resulting mixture is dried under a stream of nitrogen gas and placed in a vacuum pump until the solvent is removed. Subsequently, the samples are hydrated in a solution comprising a transition metal (which may comprise more than one metal, for example Cu and Mn, or one metal, but different salts of the metal). Preferably, the solution is buffered and metal compatible as detailed above. The mixture is then passed through an extrusion apparatus to obtain a preparation of liposomes of a defined size. Average liposome size can be determined by quasi-elastic light scattering using a NICOMP™ 370 submicron particle sizer at a wavelength of 632.8 nm. Subsequent to extrusion, the external solution may be treated or replaced so as to remove metal ions from the external solution and the liposome surface.

After formation of a liposome containing one or more encapsulated transition metals, the lactone-containing active agent is added to the extraliposomal solution and incubated at a suitable temperature to promote uptake of the drug into the liposome due to metal complexation. The above drug loading process may be carried out under conditions where the internal metal solution is unbuffered and acidic or in the presence of a buffer adjusted to the physiological pH range. This method is particularly suitable for use in the present invention as formation of the metal/active agent complex allows for stabilization of the ring-closed lactone form of the drug. This preferred technique is set forth in Example 3. As well, a second active agent may be incorporated into the liposome employing this metal-based loading technique. This method, as set forth in Example 4, involves passively entrapping an active agent along with the transition metal prior to metal loading of the lactone-containing agent. Following drug encapsulation as set forth in Examples 3 and 4, the irinotecan was analyzed by thin layer chromatography (TLC) and HPLC (Example 5) to quantify the ring-closed and ring-open forms of the drug.

The metal/active agent preparation may also be stably associated with polymeric delivery vehicles such as polymer nanoparticles, polymer microparticles, block copolymer micelles, polymer-lipid hybrid systems and derivatized single chain polymers. The preparation of these particles is described below. The use of polymers with coordination sites for complexation with metals may be included in the carriers to facilitate the loading of drugs containing lactone rings. A polymer-transition metal complex may be formed between a synthetic polymer and a metal ion via a coordinate bond. The metal is then further complexed to a lactone drug via coordination sites on the lactone ring. The introduction of only one metal binding site per polymer chain may be sufficient to promote loading of a lactone drug. The repeat units of polymer chains such as poly(acrylic acid), poly(4-vinyl pyridine), poly(L-histidine) and poly(aspartic acid) contain sites that can be coordinated to a metal ion. There are several ways in which synthetic polymers may be used to promote coordination with a lactone drug via the formation of a coordination complex. These include: 1) a homopolymer containing repeating coordination sites to complex a metal; 2) a copolymer with one block containing repeating coordination sites to complex with metal; and 3) a homopolymer with an end group containing a coordination site.

Polymer micelles are self-assembling particles composed of polymeric components that are utilized for the delivery of sparingly soluble agents present in the hydrophobic core. Various means for the preparation of micellar delivery vehicles are available and may be carried out with ease by one skilled in the art. Synthetic polymer analogs that display properties similar to lipoproteins such as micelles of stearic acid esters or poly(ethylene oxide) block-poly(hydroxyethyl-L-aspartamide) and poly(ethylene oxide)-block-poly(hydroxyhexyl-L-aspartamide) may also be used in the practice of this invention (Lavasanifar, et al., J. Biomed. Mater. Res. (2000) 52:831-835).

Nanoparticles and microparticles are polymeric delivery vehicles that comprise a concentrated core of drug that is surrounded by a polymeric shell (nanocapsules) or as a solid or a liquid dispersed throughout a polymer matrix (nanospheres). General methods of preparing nanoparticles and microparticles are described by Soppimath, et al., (J. Control Release (2001) 70(1-2):1-20) the reference of which is incorporated herein. Other polymeric delivery vehicles that may be used include block copolymer micelles that comprise a drug containing a hydrophobic core surrounded by a hydrophilic shell; they are generally utilized as carriers for hydrophobic drugs and can be prepared as found in Allen, et al., Colloids and Surfaces B: Biointerfaces (1999) Nov. 16(1-4):3-27. Polymer-lipid hybrid systems consist of a polymer nanoparticle surrounded by a lipid monolayer. The polymer particle serves as a cargo space for the incorporation of hydrophobic drugs while the lipid monolayer provides a stabilizing interference between the hydrophobic core and the external aqueous environment. Polymers such as polycaprolactone and poly(d,1-lactide) may be used while the lipid monolayer is typically composed of a mixture of lipid. Suitable methods of preparation are similar to those referenced above for polymer nanoparticles.

Derivatized single chain polymers are polymers adapted for covalent linkage of a biologically active agent to form a polymer-drug conjugate. Numerous polymers have been proposed for synthesis of polymer-drug conjugates including polyaminoacids, polysaccharides such as dextrin or dextran, and synthetic polymers such as N-(2-hydroxypropyl)methacrylamide (HPMA) copolymer. Suitable methods of preparation are detailed in Veronese and Morpurgo, IL Farmaco (1999) 54(8):497-516 and are incorporated by reference herein.

Treatment of Disease Conditions

The pharmaceutical preparations of the invention may be used to treat a variety of diseases or conditions in warm-blooded animals and in avian species. Lactone containing agents such as camptothecins are generally utilized to combat neoplasms, although the use of camptothecins to treat the skin condition, psoriasis, has been reported (Kuwahara, et al., 1985 (supra)). As well, the use of the water-soluble camptothecin derivative, topotecan, as an anti-HIV agent has been contemplated. Antibiotics are also known to contain lactone moieties and therefore preparations of the invention can be used to treat bacterial infections. The only stipulation is that the ring-closed form of the active agent is required for activity. Further examples of medical uses of the pharmaceutical preparations of the present invention include treating cardiovascular diseases such as hypertension, cardiac arrhythmia and restenosis, treating viral, fungal or parasitic infections, treating and/or preventing diseases through the use of the preparation of the present inventions as vaccines, treating inflammation or treating autoimmune diseases,

Delivery of formulated lactone agents to a tumor site is achieved by administration of delivery systems of the present invention. Preferably delivery vehicles have a diameter of less than 200 nm. Tumour vasculature is generally leakier than normal vasculature due to fenestrations or gaps in the endothelia. This allows the delivery vehicles of 200 nm or less in diameter to penetrate the discontinuous endothelial cell layer and underlying basement membrane surrounding the vessels supplying blood to a tumor. Selective accumulation of the delivery vehicles into tumor sites following extravasation leads to enhanced delivery of an encapsulated drug and therapeutic effectiveness.

Administering Pharmaceutical Preparations and Delivery Vehicles

As mentioned above, the pharmaceutical preparations of the present invention may be administered to warm-blooded animals, including humans as well as to domestic avian species. For treatment of human ailments, a qualified physician will determine how the compositions of the present invention should be utilized with respect to dose, schedule and route of administration using established protocols. Such applications may also utilize dose escalation should agents encapsulated in delivery vehicle compositions of the present invention exhibit reduced toxicity to healthy tissues of the subject.

Preferably, the pharmaceutical preparations and delivery vehicles of the present invention are administered parenterally, i.e., intraarterially, intravenously, intraperitoneally, subcutaneously, or intramuscularly. More preferably, the pharmaceutical compositions are administered intravenously or intraperitoneally by a bolus injection. For example, see Rahman, et al., U.S. Pat. No. 3,993,754; Sears, U.S. Pat. No. 4,145,410; Papahadjopoulos, et al., U.S. Pat. No. 4,235,871; Schneider, U.S. Pat. No. 4,224,179; Lenk, et al., U.S. Pat. No. 4,522,803; and Fountain, et al., U.S. Pat. No. 4,588,578.

Pharmaceutical preparations comprising delivery vehicles of the invention are prepared according to standard techniques and may comprise water, buffered water, 0.9% saline, 0.3% glycine, 5% dextrose and the like, including glycoproteins for enhanced stability, such as albumin, lipoprotein, globulin, and the like. These preparations may be sterilized by conventional sterilization techniques. The resulting aqueous solutions may be packaged for use or filtered under aseptic conditions and lyophilized, the lyophilized preparation being combined with a sterile aqueous solution prior to administration. The preparations may contain pharmaceutically acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, and the like. Additionally, the delivery vehicle suspension may include lipid-protective agents, which protect lipids against free-radical and lipid-peroxidative damages on storage. Lipophilic free-radical quenchers, such as alpha-tocopherol and water-soluble iron-specific chelators, such as ferrioxamine, are suitable.

The concentration of delivery vehicles in the pharmaceutical formulations can vary widely, such as from less than about 0.05%, usually at or at least about 2-5% to as much as 10 to 30% by weight and will be selected primarily by fluid volumes, viscosities, and the like, in accordance with the particular mode of administration selected. For example, the concentration may be increased to lower the fluid load associated with treatment. Alternatively, delivery vehicles composed of irritating lipids may be diluted to low concentrations to lessen inflammation at the site of administration. For diagnosis, the amount of delivery vehicles administered will depend upon the particular label used, the disease state being diagnosed and the judgment of the clinician.

Preferably, the pharmaceutical compositions of the present invention are administered intravenously. Dosage for the delivery vehicle formulations will depend on the ratio of drug to lipid and the administrating physician's opinion based on age, weight, and condition of the patient.

In addition to pharmaceutical compositions, suitable formulations for veterinary use may be prepared and administered in a manner suitable to the subject. Preferred veterinary subjects include mammalian species, for example, non-human primates, dogs, cats, cattle, horses, sheep, and domesticated fowl. Subjects may also include laboratory animals, for example, in particular, rats, rabbits, mice, and guinea pigs.

The following examples are offered to illustrate but not to limit the invention.

### EXAMPLES

### HPLC Analysis and Sample Preparation

Prior to HPLC analysis, samples were extracted by the addition of 100 μL aliquots to 600 μL of methanol (pre-cooled for >12 hours to −20° C.). Samples were briefly vortexed, followed by centrifugation for 10 minutes at 1500 rcf (at −8° C.). The samples were immediately analysed by HPLC. For analysis, 100 μL aliquots were loaded into 1 mL HPLC sample vials (Waters, Milford, Mass., USA) with 200 μL inserts (Chromatographic Specialities Inc., Brockville, Ont., Canada) and 10 μL were injected onto the HPLC column. The HPLC system consisted of a Model 717 plus autosampler, a Model 600E pump and controller and a Model 470 Scanning Fluorescent Detector (Waters, Milford, Mass., USA). Data were acquired and processed with the Millennium32® chromography manager (Version 3.20, Waters, Milford, Mass., USA). Separation was carried out using a Symmetry® C18 cartridge column (100 Å, particle size 5 Um; 250×4.6 mm I. D., Waters) with a Symmetry Sentry C18 guard column (particle size 5 μm; 20×3.9 mm I. D., Waters). The autosampler temperature was set to 4° C.; the column. temperature was held constant at 35° C. The mobile phase consisted of acetonitrile, 75 mM ammonium acetate, 7.5 mM tetra-butylammoniumbromide (adjusted to pH 6.4 with glacial acetic acid) (24:76, v/v), filtered trough 0.45 and 0.22 μm filters, respectively and degassed. The flow rate was 1.5 mL/min; peaks were detected at λₑₓ=362 nm (excitation wavelength) and λₑₘ=425 nm (emission wavelength). Run time was 20 min. The calibration range was 1.0 to 10.0 μg/mL for each carboxy and lactone forms of irinotecan. Calibration standards were injected both before and after extraction.

Methods for Preparation of Large Unilamellar Liposomes

Lipids were dissolved in chloroform solution and subsequently dried under a stream of nitrogen gas and placed in a vacuum pump to remove solvent. Unless otherwise specified, trace levels of radioactive lipid ³H-CHE were added to quantify lipid during the formulation process. The resulting lipid film was placed under high vacuum for a minimum of 2 hours. The lipid film was hydrated in the solution indicated to form multilamellar vesicles (MLVs). The resulting preparation was extruded 10 times through stacked polycarbonate filters with an extrusion apparatus (Lipex Biomembranes, Vancouver, BC) to achieve a mean liposome size between 80 and 150 nm. All constituent lipids of liposomes are reported in mole %.

Methods for Quantification of Drug Loading

At various time points after initiation of drug loading, aliquots were removed and passed through a Sephadex G-50 spin column to separate free from encapsulated drug. To a specified volume of eluant, Triton X-100 or N-ocyl beta-D-glucopyranoside (OGP) was added to solubilize the liposomes. Following addition of detergent, the mixture was heated to the cloud point of the detergent and allowed to cool to room temperature before measurement of the absorbance or fluorescence. Drug concentrations were calculated by comparison to a standard curve. Lipid levels were measured by liquid scintillation counting.

### Example 1

### Irinotecan Loading into Liposomes Using Metal Gluconate Salts

Liposomes have been shown to prolong the circulation lifetime of drugs in the blood and to increase accumulation at disease sites. The inventors thus examined whether the metal-drug preparations of the present invention could be stably incorporated into liposomes. The incorporation of active agents of the present invention into liposomes can be carried out by either passive or active loading techniques, although active loading is generally preferred as high levels of drug accumulation can be achieved by this method. Conventional techniques for actively loading drugs into liposomes often require the presence of a transmembrane pH gradient. These studies were performed to determine whether metal-based loading of drug could occur independently of the presence of a pH gradient by a novel active loading technique. This technique involves forming liposomes containing encapsulated metal solutions buffered to physiological pH. Following removal of external metal ions, addition of drug to the extraliposomal medium, followed by incubation at an appropriate temperature, results in drug uptake as a result of the formation of a drug-metal complex.

Experiments were conducted to examine the potential of copper, zinc and manganese gluconate to encapsulate irinotecan into liposomes. Metal gluconate solutions (100 mM) were adjusted to pH 7 using triethanolamine (TEA). The final buffer compositions were: 100 mM copper gluconate, 180 mM TEA; 100 mM manganese gluconate, 0.5 mM TEA and 100 mM zinc gluconate, 2.8 mM TEA. Lipids were weighed out (500 mg total) in order to prepare liposomes composed of DSPC/DSPG/Cholesterol (7:2:1, mol %) and were dissolved in 5 ml of dichloromethane/methanol/water (93:5:2 vol %) solvent mixture. 1 μCi of the lipid marker ¹⁴C-CHE was added to the solution and vortexed well. The solvent was transferred to a 20 ml glass reactor that was immersed in a 60° C. water bath. Various metal gluconate solutions (5 ml) were then added to the lipid-solvent mixture. The sample was mixed for 30 minutes under a stream of nitrogen gas to evaporate solvent from the system. After 30 minutes of heating, the samples were extruded, once through a 200 nm polycarbonate filter, then ten times through 2 stacked 100 nm polycarbonate filters at 70° C. To remove external copper, manganese or zinc, the samples were buffer exchanged into 300 mM sucrose, 20 mM phosphate and 10 mM EDTA, pH 7.4.

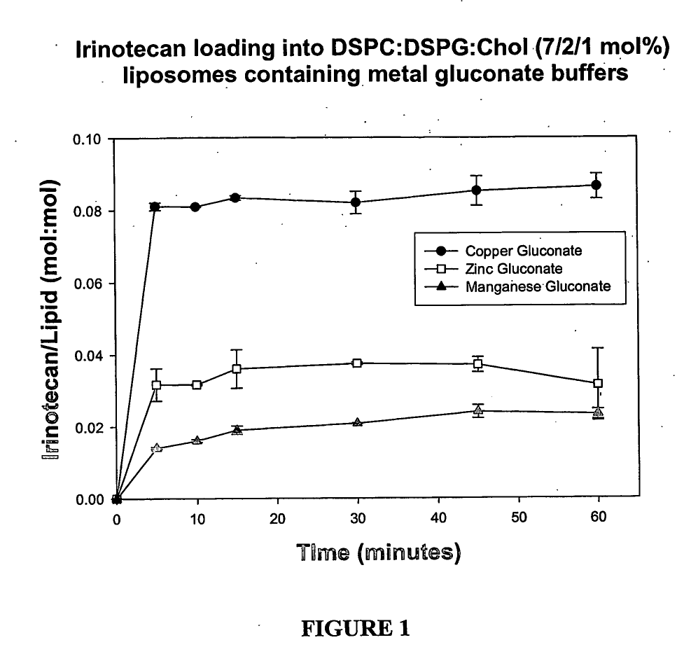

Irinotecan (20 mg) and floxuridine (100 mg) were weighed out and dissolved in 1 ml of 300 mM sucrose, 20 mM phosphate and 10 mM EDTA, pH 7.4. The solution was adjusted with 10 μl of 10M NaOH to bring the pH up to 7.0 and 10 μCi of ³H-irinotecan was also added. From this drug solution, 441 μl was added to 100 μmoles of lipid, and the sample was vortex and incubated at 50° C. At 5, 10, 15, 30, 45 and 60 minutes, 50 μl aliquots were removed from the mixture and placed on Sephadex G-50 spin columns and centrifuged at 2000 rpm for 2 minutes. This was performed in triplicate for each sample. To assay the irinotecan and lipid concentrations at each time point, liquid scintillation counting was performed using a dual-label program. The results of the loading study are plotted in FIG. 1. As shown in FIG. 1, metal-based loading of drug was accomplished with all three of the metal gluconate solutions.

### Example 2

### Metal Complexation Stabilizes the Ring-Closed Form of Camptothecins

Experiments were conducted to examine the impact of the addition of copper on the conversion of the lactone ring of irinotecan from the inactive, carboxylate form to the active, ring-closed form. The studies were carried out in the absence and presence of copper sulphate. As well, the percentage of irinotecan present in the lactone form was examined under several pH conditions as camptothecin drugs are present in the inactive hydrolyzed form at pH values around physiological pH and in the active form at low pH. The buffer composition of the preparation was also varied in order determine the effect of various buffers on the stability of the lactone moiety.

The following solutions were prepared: 1) 25 mM HEPES/150 mM NaCl; 2) 300 mM copper(II)sulphate/triethanolamine (TEA); and 3) 10% TEA/HCl. A 25 μL aliquot of a 20 mg/mL stock solution of irinotecan (hydrochloride trihydrate) were added to each of the buffers in volumetric flasks. The flasks were filled with the appropriate buffers to a volume of 50 mL to obtain final concentrations of 10 μg irinotecan/mL. The solutions were mixed, capped and stored at room temperature protected from light. At 4 hours and 24 hours after initiation of incubation, HPLC analysis of the solutions was performed according to the Methods described above to determine the lactone/carboxy ratios for irinotecan. The results are presented in Table 1 below:

TABLE 1

Incubation time

| | 4 hours | 24 hours |
|---|---|---|
| | Irinotecan lactone | Irinotecan lactone |
| Buffer (pH) | (mole % of total) | (mole % of total) |
| HEPES/NaCl (7.5) | 29.47 | 13.18 |
| HEPES/NaCl (8.5) | 7.40 | 6.44 |
| HEPES/NaCl (9.5) | 6.80 | 6.71 |
| TEA/CuSO₄ (7.5) | 56.39 | 58.66 |
| TEA/CuSO₄ (8.0) | 46.43 | 46.92 |
| TEA/CuSO₄ (8.5) | 36.16 | 38.12 |
| 10% TEA (7.5) | 21.57 | 12.39 |

The results in Table 1 show that in the absence of copper, as the pH of the irinotecan preparations increase, the carboxylate form of the drug becomes more predominate at both the 4-hour and the 24-hour time points. This trend is observed with each of the buffer solutions tested. As well, longer incubation times resulted in a lower percent of the drug being present in the ring-closed, lactone form. These results are consistent with those reported in the literature showing that the lactone/carboxylate equilibrium constant is pH dependent (Slichenmyer, et al., (supra)). In the presence of copper, after 4 hours of incubation, the lactone form of the irinotecan decreases with increases in pH. The highest levels of ring stabilization by copper were observed at pH 7.5. It is interesting to note that the optimum ring stabilization occurred at a pH that is physiologically relevant.

A comparison of the different irinotecan-containing solutions at pH 7.5 shows that at 4 hours, 56.39% of the irinotecan remains in the lactone form in the presence of CuSO₄, while only 29.47% and 21.57% of the drug is present in the active form when incubated with HEPES/NaCl and 10% TEA respectively. At 24 hours after incubation, 58.66% of the irinotecan is present in the ring-closed, lactone form, while only 13.18% and 12.39% of the drug remains in the ring-closed form when incubated in HEPES/NaCl and 10% TEA respectively at pH 7.5. These results thus demonstrate that metal ions, such as copper, can be employed to increase stabilization of the lactone ring at physiological pH.

### Example 3

### The Formulation of Metal-Camptothecin Complexes into Liposomes

In order to focus on the loading of irinotecan by copper, a liposomal formulation consisting of DSPC/DSPG/Chol (70:20:10 mole ratio) with an internal medium of copper(II)gluconate/TEA, pH 7.4 was prepared. The external pH of the formulation was 7.4 such that a transmembrane pH gradient did not exist. Lipid films of DSPC/DSPG/Chol at a mole ratio of 70:20:10 were prepared as described above in the Method section, except DSPG was dissolved in chloroform/methanol/water (50:10:1 v/v). The lipid films were hydrated in 100 mM Cu(II)gluconate, 220 mM triethanolamine (TEA), pH 7.4 and the resulting MLVs were extruded at 70° C. The liposomes were then buffer exchanged into 300 mM sucrose, 20 mM HEPES, 30 mM EDTA, pH 7.4 (SHE buffer) and then into 20 mM HEPES, 150 mM NaCl, pH 7.4 (HBS buffer) by tangential flow dialysis. Irinotecan was added to the liposome preparation at a 0.1:1 drug-to-lipid mole ratio and the extent of drug loading was determined, as described in the Methods, by measuring irinotecan absorbance at 370 nm and lipid levels by liquid scintillation counting.

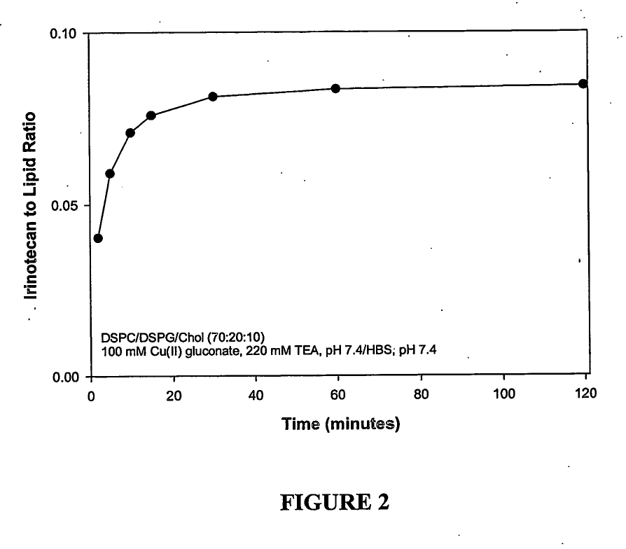

Results depicted in FIG. 2 show the uptake of irinotecan into DSPC/DSPG/Chol (70:20:10 mole ratio) liposomes as a function of time. Loading of irinotecan occurred efficiently during the time course measured. These results demonstrate that metal-based loading of a camptothecin drug, such as irinotecan, can be achieved without a pH gradient. This alleviates the need to employ low pH values commonly utilized with liposomal formulations to stabilize the lactone ring. As described previously, this method overcomes various limitations associated with the presence of a low intraliposomal pH, such as lipid hydrolysis and poor pH gradient loading of camptothecin drugs. A further advantage of this loading technique is that it results in the co-encapsulation of a camptothecin drug and a transition metal thereby allowing for the stabilization of the camptothecin by copper at a pH in which the drug would normally equate to the inactive, carboxylate form.

Copper loading of irinotecan into cholesterol-containing liposomes without a pH gradient was also investigated employing DPPC/Chol (55:45 mole ratio) liposomes. The liposomes were prepared as described in the methods by hydrating lipid films in a solution of 100 mM copper(II)gluconate adjusted to pH 7.4 with TEA. Liposomes were extruded at 65° C. and the external buffer of the liposomes was exchanged to SHE, pH 7.4 by tangential flow dialysis. Liposomes were incubated with irinotecan at a 0.1:1. drug-to-lipid weight ratio at 50° C. and the extent of drug loading was determined as described by measuring irinotecan absorbance at 370 nm after solubilization by detergent.

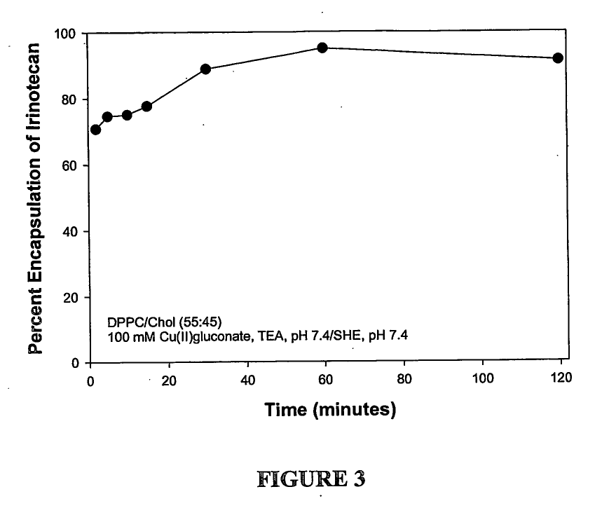

Loading of irinotecan into DPPC/Chol (55:45 mole ratio) liposomes in the absence of a pH gradient revealed that almost complete loading was observed after about 60 minutes of incubation (FIG. 3). These results thus demonstrate that metal-based loading of irinotecan can be achieved using various liposomal formulations.

### Example 4

### Metal Loading of a Camptothecin Drug into Buffered Liposomes Containing a Passively Encapsulated First Drug

Although Example 3 describes the metal-induced loading of one drug into liposomes, the technique can be employed to load two or more drugs into a single liposome. This allows for the preparation of liposomes containing two or more therapeutic agents that can be used to treat disease resulting from multiple molecular mechanisms, such as cancer. One technique of loading two agents into a liposome involves first passively entrapping at least one drug along with a metal followed by active metal loading of the camptothecin drug. In this example, liposomes with co-encapsulated with irinotecan and floxuridine (FUDR) by first passively entrapping FUDR along with copper. The FUDR loaded liposomes were subsequently loaded with irinotecan by metal loading according to the technique described in Example 3. The internal and external buffer solutions were adjusted to pH 7.4 thus ensuring that the second drug was encapsulated by metal loading.

Entrapment of irinotecan into DSPC/DSPG/Chol (70:20:10 mole ratio) and DSPC/DSPG (85:15 mole ratio) liposomes, containing passively encapsulated floxuridine (FUDR), was carried out by dissolving DSPC and cholesterol (if present) in chloroform and DSPG in chloroform/methanol/water (50:10:1 v/v). The lipids were then combined together at the specified mole ratios and labeled with trace amounts of ¹⁴C-CHE. The samples were hydrated in 100 mM copper(II)gluconate, 220 mM TEA, pH 7.4, containing 100 mM FUDR with trace levels of ³H-FUDR. The resulting MLVs were extruded at 70° C., then buffer exchanged first into saline and next into SHE, pH 7.4 using a hand-held tangential flow dialysis column to remove Cu(II)gluconate and unencapsulated FUDR. The samples were then further exchanged into 300 mM sucrose, 20 mM HEPES, pH 7.4 to remove any EDTA in the exterior buffer. Irinotecan was added to the resulting liposome preparation at a drug-to-lipid mole ratio of0.1:1 for DSPC/DSPG liposomes and 0.09:1 for DSPC/DSPG/Chol liposomes. As described in the Methods, a drug-to-lipid ratio for the spun column eluant was generated using liquid scintillation counting to determine lipid and FUDR concentrations, and absorbance at 370 nm to determine irinotecan concentrations.

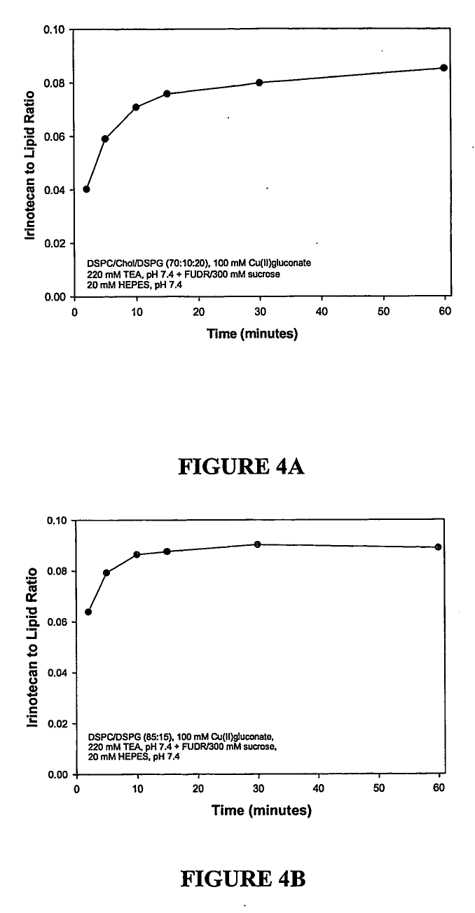

FIG. 4A shows that loading of irinotecan into DSPC/DSPG/Chol (70:20:10 mole ratio) liposomes containing encapsulated FUDR and metal does not require the presence of a pH gradient as efficient loading of the drug occurred throughout the time course of the experiment. Similarly, results summarized in FIG. 4B show that irinotecan efficiently loads into DSPC/DSPG (85:15 mole ratio) liposomes with encapsulated FUDR. These results thus demonstrate that liposomes containing a camptothecin drug along with a second drug, such as a pyrimidine derivative, can be prepared employing metal-based loading procedures coupled with passive loading.

### Example 5

### Analysis of Liposomal Irinotecan for the Distribution of Carboxylate and Lactone Species

Three sets of liposomal formulations of irinotecan were prepared as previously described. In one formulation (A) the irinotecan was loaded into liposomes containing 300 mM copper sulfate, 20 mM HEPES and pH adjusted to 7.5 with triethanolamine. Irinotecan was added to the external liposomal buffer and incubated at 50° C. to promote drug encapsulation. Any unencapsulated irinotecan was removed by column chromatography. Another liposomal formulation (C) contained 100 mM copper gluconate and 180 mM triethanolamine (pH 7) and loaded with irinotecan as previously described in Example 3. The third formulation (B) contained irinotecan and floxuridine in the final composition. In this formulation the liposomes were prepared in the presence of floxuridine and then unencapsulated drug was removed by chromatography. Irinotecan was subsequently loaded into the formulation as described in Example 4. In all cases the external pH was adjusted to match the internal liposomal pH prior to irinotecan loading.

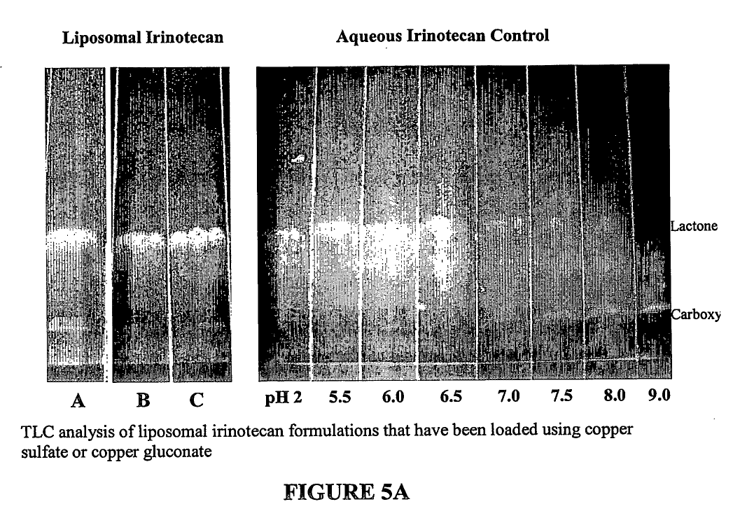

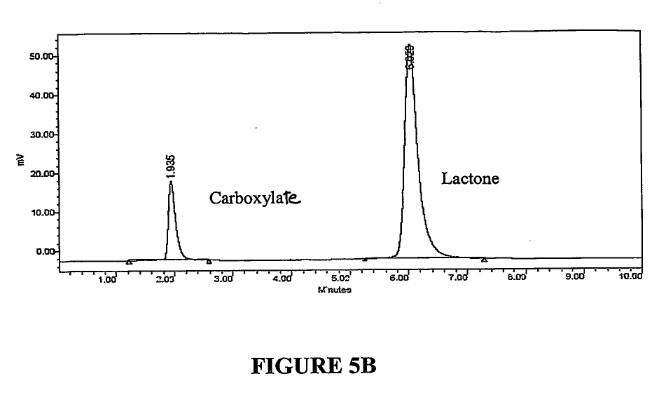

Liposomal formulations of irinotecan were first analyzed for ring closed lactone and ring open carboxylate forms of the drug by thin layer chromatography. A set of standards were first prepared and used as a reference for the liposomal formulations. The aqueous standards were prepared by diluting an irinotecan stock solution into HEPES buffered saline solutions that were pH adjusted between 2 and 9 with NaOH or HCl. The irinotecan was left in these buffered solutions for 30 minutes and then extracted into a chloroform:methanol (1:1) solution. Approximately 50 ng of irinotecan was loaded onto the origin of silica gel 60 hard TLC plates and run in the solvent system composed of chloroform:methanol:acetone (9:3:1). The TLC plates were dried overnight at room temperature and then run in a second solvent system composed of butanol:acetic acid:water:acetone (4:2:1:1). The irinotecan control bands were subsequently visualized under UV light (FIG. 5A). As expected, under acidic conditions, the lactone form of irinotecan is the dominant species. As the pH of the solution increased, the presence of the carboxylate band appears.

For the analysis of liposomal formulations, samples were diluted into chloroform:methanol (1:1) and 50 ng of irinotecan loaded onto the TLC plate. The plates were run in the same solvent systems and visualized by UV light. In all three formulations (A, B and C), the majority of the irinotecan was isolated as the closed-ring lactone species even though the internal buffer was pH 7 or 7.5. Lane A contains liposomes containing 300 mM copper sulfate, 20 mM HEPES and pH adjusted to 7.5 with triethanolamine that were loaded with irinotecan at a drug to lipid ratio of 0.2/1 (mol:mol). Lane B contains liposomes containing floxuridine were loaded with irinotecan using 100 mM copper gluconate and 180 mM TEA (pH 7.0) as an internal buffer and 300 mM sucrose, 40 mM phosphate (pH 7.0) as an external buffer. Lane C contains liposomes containing 100 mM copper gluconate and 180 mM TEA (pH 7.0) that were loaded with irinotecan at a drug to lipid ratio of 0.1/1.

The liposomal formulations prepared in lanes A and C were also extracted and separated by HPLC to quantify the lactone and carboxylate percentages.

Irinotecan was separated on a C18 column using a mobile phase of 78% (3% triethanolamine solution) and 22% acetonitrile. The sample was quantified using a fluorescence detector with an excitation wavelength of 363 nm and an emission of 425 nm. The relative percentages of lactone and carboxylate were based on the peak area generated with an irinotecan standard. The results from the copper sulfate formulation (Lane A) are shown in FIG. 5B. Based on an irinotecan standard the relative percentages were determined to be 83% lactone and 17% carboxylate. The results from copper gluconate (Lane C) were determined to be 90% lactone and 10% carboxylate.

Both liposomal formulations show a high percentage of the irinotecan in the lactone form. The results of the liposomal formulation analysis by TLC and HPLC support the observation of Example 1 where the presence of copper dramatically enhances the presence of the lactone species of irinotecan.

We claim:

1. A pharmaceutical composition comprising an active agent having a lactone ring and a transition metal ion, wherein said ion is present at sufficient concentration to stabilize said lactone.

2. The composition of claim 1 wherein the pH of the preparation is between 6.0 and 8.0.

3. The composition of claim 1 wherein at least 40 mole % of the active agent is present in the ring-closed, lactone form at physiological pH.

4. The composition of claim 1 wherein at least 50 mole % of the active agent is present in the ring-closed, lactone form at physiological pH.

5. The composition of claim 1 wherein the active agent is camptothecin or a related analog.

6. The composition of claim 5 wherein the camptothecin is a water-soluble analog.

7. The composition of claim 6 wherein the water-soluble analog is selected from the group consisting of topotecan, irinotecan and lurtotecan.

8. The composition of claim 1 wherein the active agent and the metal are at a concentration of greater than 100 μM.

9. The composition of claim 1 wherein the transition metal complexes with the active agent through the oxygen coordination sites on the lactone ring.

10. The composition of claim 1 wherein said ion is of transition metal is selected from the group consisting of Cu, Zn and Co.

11. The composition of claim 10 wherein the transition metal is Cu.

12. The composition of claim 1 wherein the active agent and the transition metal ion are stably associated with one or more delivery vehicles.

13. The composition of claim 12 wherein the delivery vehicle is selected from the group consisting of lipid carriers, liposomes, lipid micelles, lipoprotein micelles, lipid-stabilized emulsions, cyclodextrins, polymer nanoparticles, polymer microparticles, block copolymer micelles, polymer-lipid hybrid systems and derivatized single chain polymers.

14. The composition of claim 13 wherein the delivery vehicle is a liposome.

15. The composition of claim 14 wherein the liposome is a large unilamellar liposome.

16. The composition of claim 12 wherein the delivery vehicle is a liposome and the transition metal ion is Cu+2.

17. The composition of claim 12 wherein the active agent is a camptothecin.

18. The composition of claim 17 wherein the camptothecin is a water-soluble analog selected from the group consisting of lurtotecan, topotecan and irinotecan.

19. The composition of claim 13 wherein the delivery vehicle is a polymer nanoparticle.

20. The composition of claim 19 wherein one or more polymers making up the nanoparticle are complexed with a transition metal ion.

21. The composition of claim 20 wherein the nanoparticle comprises a stabilizing lipid.
